(19) United States
(12) Patent Application Publication
(10) Pub. No.: US 2008/0275389 A1
(43) Pub. Date: Nov. 6, 2008

(54) Catheter Handle and a Catheter Assembly Including Such a Handle

(76) Inventors: Zoran Milijasevic, Bayview (AU); Neil Lawrence Anderson, Roseville (AU); Evan Chong, South Strathfield (AU)

Correspondence Address: MORRISON & FOERSTER LLP, 755 PAGE MILL RD, PALO ALTO, CA, 94304-1018, US

(21) Appl. No.: 11/885,025

(22) PCT Filed: Mar. 1, 2006
(86) PCT No.: PCT/AU06/00268

§ 371 Date: Jul. 9, 2008

Related U.S. Application Data
(60) Provisional application No. 60/658,753, filed on Mar. 4, 2005.

(51) Int. Cl. A61M 25/092; A61F 7/12

(52) U.S. Cl. 604/95.04; 607/113

(57) ABSTRACT

A catheter handle (10) includes a handle body (12) having a proximal end (14) and a distal end (16) with a receiving formation (18) being defined at the distal end (16) for receiving a catheter arrangement. A steering control mechanism (22) is carried by the handle body (12), a steering device of the catheter arrangement being connectable to the steering control mechanism (22). At least one introducer zone (24) is defined by a part of the handle body (12) and is in communication with the distal end (16) of the handle body (12). A shape forming element is able to be introduced, in use, via the introducer zone (24) into a lumen of a catheter of the catheter arrangement received by the receiving formation (18).

### CROSS-REFERENCE TO RELATED APPLICATIONS

The present application claims priority from U.S. Provisional Patent Application No. 60/658,753 filed on 4 Mar. 2005, the contents of which are incorporated herein by reference.

### FIELD OF THE INVENTION

This invention relates, generally, to the field of catheters and, more particularly, to a catheter handle, to a catheter assembly including such a handle and to a method of delivering a catheter to a biological site in a patient's body.

### BACKGROUND TO THE INVENTION

In the heat treatment of a biological site in a patient's body, either surface irregularities at the site or the accessibility and conformity of the catheter to various body regions may impede electrode-tissue contact. This could result in the treatment regime not being as efficient as would otherwise be the case. In order to overcome or accommodate such surface irregularities, it would be useful to be able to apply pressure on the electrode, or electrodes, being used to carry out the heat treatment.

The Applicant has filed an International Patent Application for an electrical lead under International Patent Application Number PCT/AU01/01339 dated 19 Oct. 2001. The contents of that application are incorporated in this specification by reference. The electrical lead forming the subject matter of the International Patent Application is suitable as an electrode sheath of a catheter to be used in the heat treatment of a biological site in a patient's body. It lends itself to this application due to the fact that such an electrode sheath has an unimpeded lumen into which items to assist in maneuvering and manipulating the electrode sheath can be inserted while retaining a narrow diameter sheath which is beneficial in steering the catheter through the vascular system of the patient to the site of interest.

### SUMMARY OF THE INVENTION

According to a first aspect of the invention, there is provided a catheter handle which includes:

a handle body having a proximal end and a distal end with a receiving formation being defined at the distal end for receiving a catheter arrangement;

a steering control mechanism carried by the handle body, a steering device of the catheter arrangement being connectable to the steering control mechanism; and

at least one introducer zone defined by a part of the handle body and in communication with the distal end of the handle body, a shape forming element being able to be introduced, in use, via the introducer zone into a lumen of a catheter of the catheter arrangement received by the receiving formation.

The introducer zone may comprise at least one introducer port angled with respect to, and intersecting, the receiving formation at the distal end of the handle body. A guide formation may be arranged at a proximal opening to the introducer port to facilitate the insertion of a shape forming element into a lumen of a catheter of the catheter arrangement.

Preferably, the catheter handle is intended for use with a catheter arrangement comprising at least two catheters carried in a tube, a proximal end of a passage defined in the handle body defining a corresponding number of seats to the number of catheters of the catheter arrangement, in each of the seats an electrical connector being receivable for interconnecting an electrical control unit and electrodes of the catheters. For example, the proximal end of the passage of the handle body may be bifurcated to define two seats. An electrical connector may be received in each seat for interconnecting the electrical control unit and the electrodes of the catheters.

The catheter handle may include an introducer port associated with each of at least certain of the catheters of the catheter arrangement, the introducer ports being arranged in circumferentially spaced relationship about the receiving formation of the handle body. Thus, for example, where the catheter arrangement comprises two catheters, the introducer ports may be arranged on opposed sides of the receiving formation.

The receiving formation at the distal end of the handle body may be in the form of an axially extending bore in which a proximal end of the catheter arrangement is receivable, a proximal end of the bore opening into the passage of the handle body.

The steering control mechanism may comprise an assembly that acts on at least one steering element, such as a steering wire, of the steering device of the catheter arrangement.

The assembly may be a slide assembly. The slide assembly may comprise at least one slider with the steering wire of the steering device of the catheter arrangement being connectable to the at least one slider to effect bending of a distal end of the catheter arrangement by manipulation of the slide assembly. The at least one slider may therefore include a boss to which a proximal end of the associated steering wire is connectable.

In one embodiment, the slide assembly may include two sliders which are independently moveable relative to the handle body. In this embodiment, manipulation of the sliders causes bending of a distal end of the tube of the catheter arrangement in-plane.

In another embodiment, the slide assembly may include at least three sliders, each of which has a steering wire connected to it, in use. Distal ends of the steering wires may be connected at circumferentially, equi-spaced points at a distal end of the tube. Then, by manipulating any two of the sliders with respect to a remainder of the sliders, omni-directional steering of the distal end of the tube of the catheter arrangement may be effected.

According to a second aspect of the invention, there is provided a catheter assembly which includes:

a handle body having a proximal end and a distal end with a receiving formation being defined at a distal end of the handle body, a steering control mechanism carried by the handle body and at least one introducer zone in communication with the receiving formation of the handle body;

a catheter arrangement having a proximal end received in the receiving formation of the handle body, the catheter arrangement comprising at least two catheters each having an electrode sheath, distal ends of the electrode sheaths being interconnected to define a bridging portion; and

at least one shape forming element received through the at least one introducer zone into a lumen of at least one of the catheters to impart a predetermined shape to the bridging portion of the catheter arrangement.

The bridging portion of the catheter arrangement may carry heat treating electrodes which are able to be urged into contact with tissue at a biological site in a patient's body for effecting heat treatment at the site. For example, the electrodes may be ablating electrodes for the treatment of heart arrhythmias by the formation of one or more lesions at the biological site.

It will be appreciated that, with the provision of two separate catheters and the bridging portion of their electrode sheaths, pressure can be exerted on the bridging portion, suitably stiffened by the shape forming element, to urge the electrodes into contact with the tissue at the biological site.

To facilitate introduction of the catheter arrangement into a vascular system of a patient to be treated, the electrode sheaths may be contained within a tube which, in turn, is received, in use, in an introducer for introduction into the vascular system of the patient for delivery of a distal part of the catheter arrangement to the biological site.

The tube may have a plurality of steering components, such as a steering wire, for effecting steering of a distal end of the tube. The steering control mechanism of the handle body may comprise a plurality of steering members, such as sliders, with a proximal end of each steering wire being connected to one of the steering members for facilitating steering of the distal end of the tube.

The distal end of the electrode sheath of each catheter may carry a plurality of axially spaced electrodes.

According to a third aspect of the invention, there is provided a method of delivering a catheter to a biological site in a patient's body, the method including:

inserting a catheter arrangement into an introducer, the catheter arrangement comprising at least two catheters having electrode sheaths interconnected at their distal ends, the catheters being arranged in a steerable tube;

inserting the introducer into the vascular system of the patient and steering a distal end of the introducer to the site;

inserting a shape forming element into a lumen of at least one of the electrode sheaths to form a bridging portion of a predetermined shape at the distal end of the catheter arrangement; and

urging the interconnected distal ends of the electrode sheaths out of a distal end of the introducer.

The method may include, initially, inserting a stiffening element into a lumen of at least one of the electrode sheaths to facilitate steering of the catheter arrangement to the site, the stiffening element being in the form of a straight stylet.

Once a distal end of the introducer is at the biological site, the method may include removing the stiffening element and replacing it with at least one shape forming stylet.

Further, the method may include, once the bridging portion of the catheter is exposed at the site, applying pressure to the bridging portion to urge electrodes carried by the bridging portion into contact with tissue at the site to enhance electrode-tissue contact.

### BRIEF DESCRIPTION OF THE DRAWINGS

FIG. 1 shows a three dimensional view of a catheter handle, in accordance with an embodiment of the invention;

FIG. 2 shows, on an enlarged scale, a three dimensional view of a part of a distal part of the catheter handle;

FIG. 3 shows a three dimensional view of the part of the catheter handle of FIG. 2 with a steering control mechanism of the catheter handle omitted;

FIG. 4 shows, on a further enlarged scale, a three dimensional view of a slider of the steering control mechanism of the catheter handle;

FIG. 5 shows, on a still further enlarged scale, a three dimensional view of a guide element of an introducer zone of the catheter handle; and

FIG. 6 shows a three dimensional view of a catheter assembly, in accordance with a further embodiment of the invention.

### DESCRIPTION OF EXEMPLARY EMBODIMENT

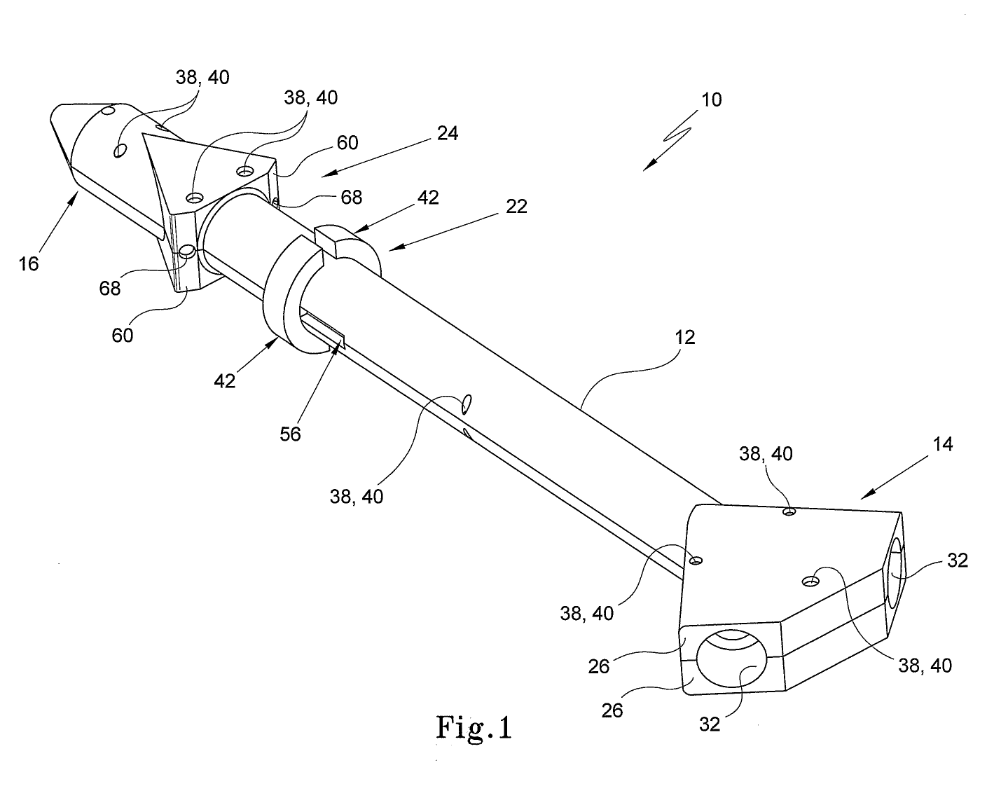

Referring initially to FIGS. 1 to 5 of the drawings, reference numeral 10 generally designates a catheter handle in accordance with an embodiment of the invention.

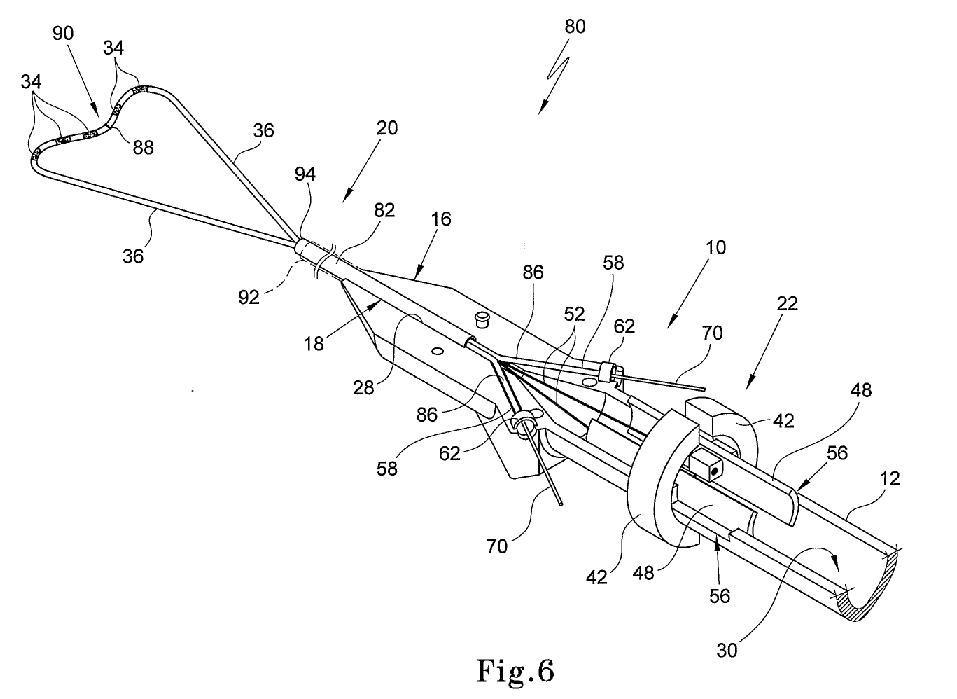

The catheter handle 10 comprises an elongate handle body 12. The handle body 12 has a proximal end 14 and a distal end 16. A receiving formation 18 is defined at the distal end 16 for receiving a catheter arrangement 20 (FIG. 6). A steering control mechanism 22 is carried by the handle body 12 proximal the distal end 16 of the handle body 12. The handle 10 includes an introducer zone 24 arranged intermediate the steering control mechanism 22 and the distal end 16 of the handle body 12.

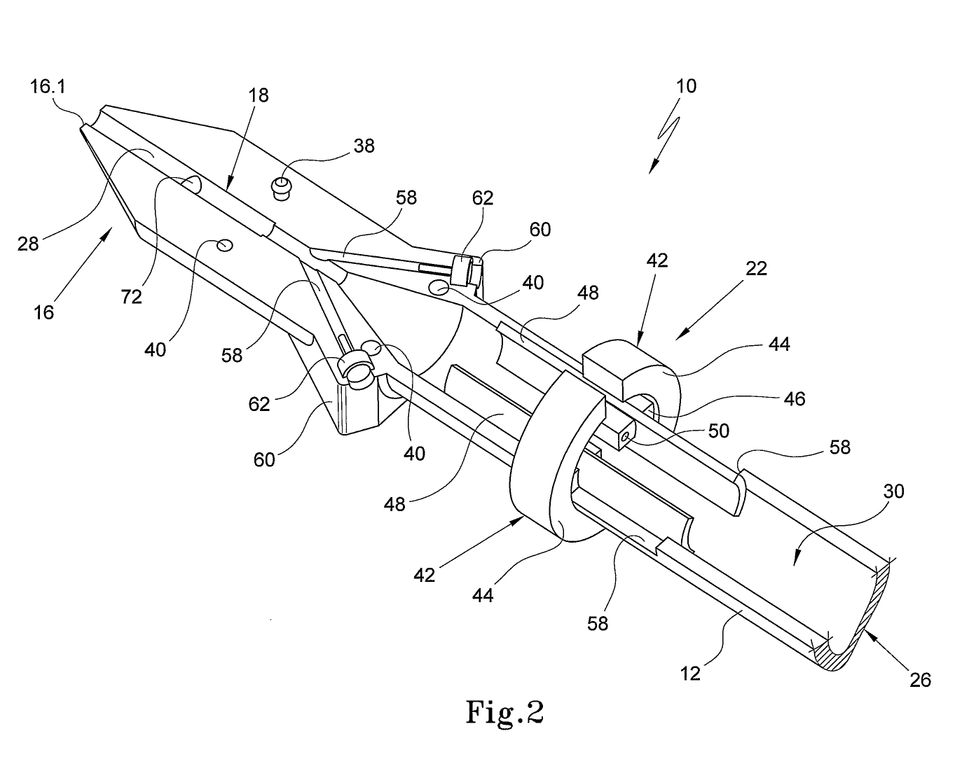

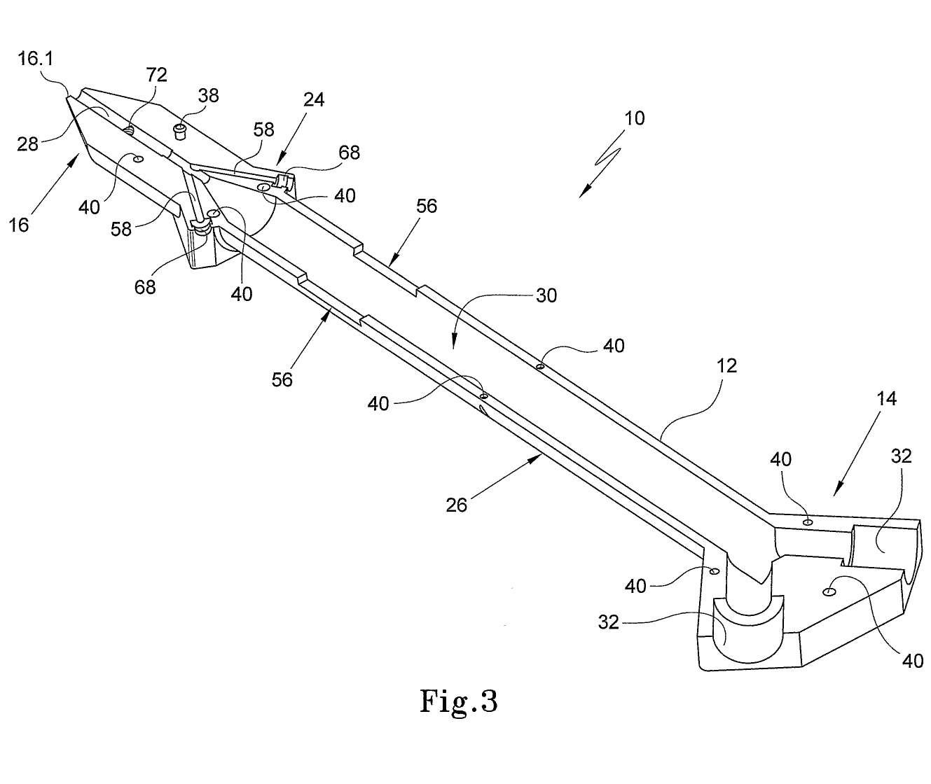

The handle body 12 comprises a pair of shells 26. A distal part of one of the shells 26 is shown in greater detail in FIGS. 2 and 3 of the drawings.

The receiving formation 18 is in the form of an axially extending bore 28 opening out into a distal tip 16.1 of the distal end 16 of the handle body 12. A proximal end of the bore 28 opens out into an axially extending passage 30.

As shown in greater detail in FIG. 1 of the drawings, a proximal end of the passage 30 is bifurcated and defines a pair of seats 32 at the proximal end 14 of the handle body 12. An electrical connector (not shown) is received in each of the seats 32 and communicates with electrodes 34 (FIG. 6) of catheters 36 of the catheter arrangement 20 via electrical leads (not shown) extending along the passage 30 to the distal end 16 of the handle body 12.

The two shells 26 are secured together via complementary pins 38 and sockets 40 arranged at predetermined locations on mating lands of the shells 26.

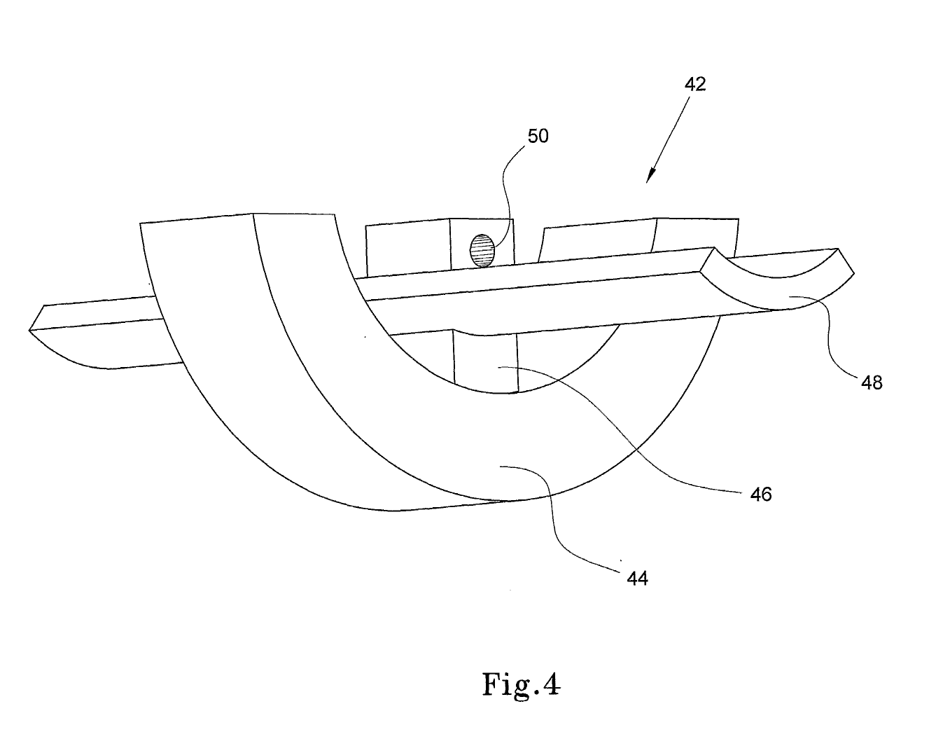

The steering control mechanism 22 comprises a slide assembly constituted by a pair of sliders 42. One of the sliders 42 is shown in greater detail in FIG. 4 of the drawings. Each slider 42 includes a manipulating element in the form of a semi-annulus 44 arranged outwardly of the handle body 12. A boss 46 projects radially inwardly from an inner surface of the semi-annulus 44. The boss 46 supports a slide element 48. The slide element 48 is received in the passage 30 of the handle body 12 and serves to inhibit the ingress of foreign material into the passage 38 of the handle body 12.

A bore 50 is defined in a part of the boss 46 inwardly of the slide element 48. A steering component, in the form of a steering wire 52 (FIG. 6), is secured to its associated slider 42 by being received in the bore 50 and secured at a proximal side of the boss 46.

The part of the boss 46 between the semi-annulus 44 and the slide formation 48 is received in a slot 56 extending axially in a wall of the handle body 12. A part of each slot is defined by each shell 26 so that when the shells 26 are mated together, a pair of opposed, completed slots 56 results. Each slot 56 holds one of the sliders 42 displaceably captive relative to the handle body 12. A part of each slot 56 is shown in greater detail in FIG. 3 of the drawings.

The introducer zone 24 comprises two introducer ports 58 arranged on opposed sides of the passage 28 and intersecting a proximal region of the passage 28. The ports 58 are defined in a backswept wing member 60 of the handle body 12.

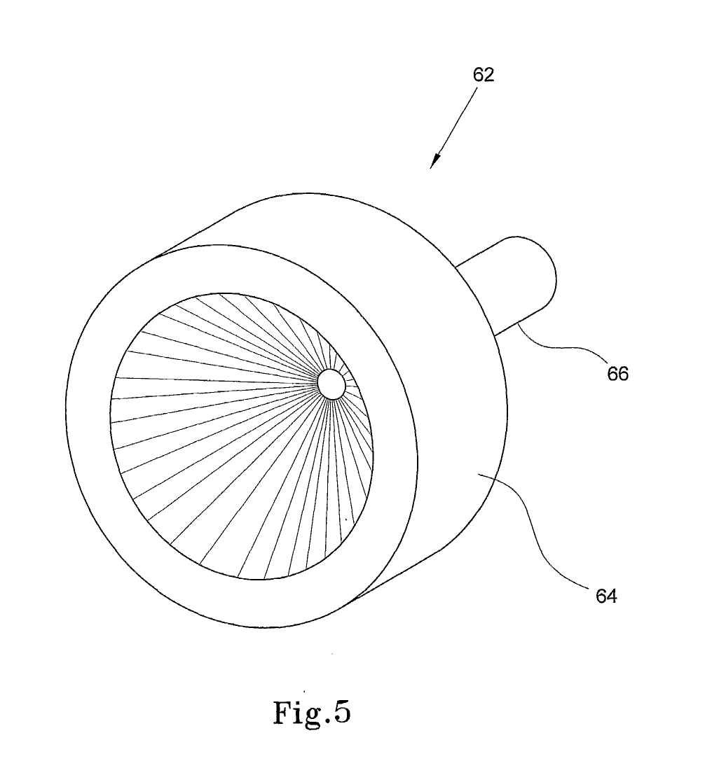

A guide formation 62 is arranged at a proximal end of each introducer port 58. One of the guide formations 62 is shown in greater detail on a considerably enlarged scale in FIG. 5 of the drawings. Each guide formation 62 has a funnel shaped element 64 opening out into a sleeve 66. The funnel shaped element 64 is received in a seat 68 (FIG. 3) at a proximal opening of its associated introducer port 58.

The funnel shaped element 64 of the guide formation 62 serves to guide a distal end of a shape forming element, or stylet, 70 (FIG. 6) into a lumen of its associated catheter 36 of the catheter arrangement 20.

As shown more clearly in FIGS. 2 and 3 of the drawings, each shell 26 has a transversely extending hole 72 which intersects the passage 28. Each hole 72 is threaded. A retaining formation, in the form of a grub screw (not shown), is received in each hole 72 for retaining a proximal end of the catheter arrangement 20 in position relative to the handle 10.

Referring now to FIG. 6 of the drawings, a catheter assembly 80, in accordance with another embodiment of the invention is described. The catheter assembly 80 includes the catheter handle 10 with a catheter arrangement 20 secured to a distal end 16 of the handle 10. The catheter arrangement 20 includes a tube 82 containing the steering wires 52. A proximal end of the tube 82 is received in the passage 28 of the receiving formation 18 of the handle 10. The tube 82 has a passage which accommodates two catheters 36. Each catheter 36 has an electrode sheath 86 manufactured in accordance with the Applicant's International Application No. PCT/AU01/01339, referenced above. An electrode sheath 86 manufactured in accordance with this manufacturing technique has an unimpeded lumen due to electrical conductors for the electrodes 34 being contained in a wall of the electrode sheath 86. These conductors (not shown) extend along the passage 30 of the handle 10 to electrical connectors contained in the seats 32 at the proximal end 14 of the handle 10 to effect electrical interconnection between the electrodes 34 and an electrical control unit (not shown) to which the assembly 80 is connected for use.

A proximal end of each sheath 86 is received over the sleeve 66 of the guide formation 62 at the proximal end of its associated introducer port 58. This facilitates the insertion of the shape forming element 70 into the lumen of the catheter sheath 86 of the catheter 36.

Distal ends of the electrode sheaths 86 of the catheters 36 are connected together as shown at 88 to form a bridging portion 90.

Initially, to deliver a distal part of the catheter arrangement 20 to a biological site, such as an atrium of a patient's heart, the catheter arrangement 20 is inserted into an introducer 92 with a distal part of the catheter arrangement 20 being withdrawn into a distal end of the introducer 92. The introducer 92 is inserted into a femoral vein of the patient's body and is steered through the vascular system of the patient until a distal part of the introducer 92 is in proximity to the site to be treated.

The distal part of the catheter arrangement 20 is then urged out of the distal end of the introducer 92 so that the bridging portion 90 of the catheter arrangement 20 is free of the introducer 92.

To facilitate steering of the distal part of the tube 82 and the catheter arrangement 20 through the vascular system, straight stylets are used, initially, as the shape forming elements 70.

The distal part of the introducer 92 is steered via the distal part of the tube 82. Thus, distal ends of the steering wires 52 of the tube 82 have their distal ends connected to a distal end 94 of the tube 82 in a diametrically opposed relationship. By appropriate manipulation of the sliders 42 of the steering control mechanism 22 axially with respect to each other in-plane, steering of the distal part of the tube 82 and, accordingly, the introducer 92 is effected.

When the distal part of the introducer 92 is in proximity to the site to be treated and before the catheter arrangement 20 is urged out of the introducer 92, the straight stylets are withdrawn from the lumens of the electrode sheaths 86 of the catheters 36. A stylet 70 of a predetermined shape is then inserted into the lumen of each electrode sheath 86 up to the junction 88 in the bridging portion 90. After insertion of the stylets 70 has been completed, the distal part of the catheter arrangement 20 is urged out of the distal end of the introducer 92. The stylets 70 impart a predetermined shape to the bridging portion 90 of the catheter arrangement 20 such as, for example, an S-shaped bridging portion 90 or an undulating bridging portion 90. It will therefore be appreciated that the stylets 70 are of a superlastic alloy, such as nitinol, preformed into the desired shape.

Once the bridging portion 90 is free of the introducer 92, the bridging portion 90 is urged into contact with tissue at the site. Heat treatment of the tissue can then be effected by energising the electrodes 34. For example, in the treatment of heart arrhythmias, the electrodes 34 ablate the tissue to form lesions in an attempt to eradicate the arrhythmia.

It is therefore desirable that intimate electrode-tissue contact occur. To facilitate this, and in an endeavour to overcome surface irregularities at the site being treated, pressure can be applied to the bridging portion 90 by means of the handle 10 and the inherent stiffness of the stylets 70. The use of appropriate stylets 70 in the bridging portion 90 may also enhance accessibility at difficult to access locations at the site being treated.

It is therefore an advantage of the invention that a catheter assembly 80 is provided which aids in overcoming tissue surface irregularities at a biological site to be treated. Further, the catheter assembly 80 may, by appropriate choice of stylets 70, improve accessibility to awkward to access locations at the site being treated. It is a further advantage of the invention that, due to the use of two catheters 36, double the number of electrodes are provided than would otherwise be the case. While only five electrodes 34 have been shown, this is purely for illustration purposes and the electrode sheath 86 of each catheter 36 could carry up to ten electrodes. It will be appreciated that, with this configuration, very fine control of lesion formations can be effected.

It is yet a further advantage of the invention that, with the provision of the introducer ports 58, it is a reasonably straight forward procedure for a clinician to replace stylets 70 of one shape with stylets 70 of a different shape to provide further fine control of the formation of lesions at the site.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1: A catheter handle which includes:
a handle body having a proximal end and a distal end with a receiving formation being defined at the distal end for receiving a catheter arrangement;
a steering control mechanism carried by the handle body, a steering device of the catheter arrangement being connectable to the steering control mechanism; and
at least one introducer zone defined by a part of the handle body and in communication with the distal end of the handle body, a shape forming element being able to be introduced, in use, via the introducer zone into a lumen of a catheter of the catheter arrangement received by the receiving formation.

2: The catheter handle of claim 1 in which the introducer zone comprises at least one introducer port angled with respect to, and intersecting, the receiving formation at the distal end of the handle body.

3: The catheter handle of claim 2 in which a guide formation is arranged at a proximal opening to the introducer port to facilitate the insertion of a shape forming element into a lumen of a catheter of the catheter arrangement.

4: The catheter handle of claim 2 which is intended for use with a catheter arrangement comprising at least two catheters carried in a tube, a proximal end of a passage defined in the handle body defining a corresponding number of seats to the number of catheters of the catheter arrangement, in each of the seats an electrical connector being receivable for interconnecting an electrical control unit and electrodes of the catheters.

5: The catheter handle of claim 4 which includes an introducer port associated with each of at least certain of the catheters of the catheter arrangement, the introducer ports being arranged in circumferentially spaced relationship about the receiving formation of the handle body.

6: The catheter handle of claim 4 in which the receiving formation at the distal end of the handle body is in the form of an axially extending bore in which a proximal end of the catheter arrangement is receivable, a proximal end of the bore opening into the passage of the handle body.

7: The catheter handle of claim 1 in which the steering control mechanism comprises an assembly that acts on at least one steering element of the steering device of the catheter arrangement.

8: The catheter handle of claim 7 in which the assembly is a slide assembly.

9: The catheter handle of claim 8 in which the slide assembly comprises at least one slider with a component of the steering device of the catheter arrangement being connectable to the at least one slider to effect bending of a distal end of the catheter arrangement by manipulation of the slide assembly.

10. A catheter assembly which includes:
a handle body having a proximal end and a distal end with a receiving formation being defined at a distal end of the handle body, a steering control mechanism carried by the handle body and at least one introducer zone in communication with the receiving formation of the handle body;
a catheter arrangement having a proximal end received in the receiving formation of the handle body, the catheter arrangement comprising at least two catheters each having an electrode sheath, distal ends of the electrode sheaths being interconnected to define a bridging portion; and
at least one shape forming element received through the at least one introducer zone into a lumen of at least one of the catheters to impart a predetermined shape to the bridging portion of the catheter arrangement.

11: The assembly of claim 10 in which the bridging portion of the catheter arrangement carries heat treating electrodes which are able to be urged into contact with tissue at a biological site in a patient's body for effecting heat treatment at the site.

12: The assembly of claim 11 in which the electrode sheaths are contained within a tube which, in turn, is, in use, received in an introducer for introduction into the vascular system of the patient for delivery of a distal part of the catheter arrangement to the biological site.

13: The assembly of claim 12 in which the tube has a plurality of steering components for effecting steering of a distal end of the tube.

14: The assembly of claim 13 in which the steering control mechanism of the handle body comprises a plurality of steering members with a proximal end of each steering component being connected to one of the steering members for facilitating steering of the distal end of the tube.

15: The assembly of claim 9 in which the distal end of the electrode sheath of each catheter carries a plurality of axially spaced electrodes.

16: A method of delivering a catheter to a biological site in a patient's body, the method including:
inserting a catheter arrangement into an introducer, the catheter arrangement comprising at least two catheters having electrode sheaths interconnected at their distal ends, the catheters being arranged in a steerable tube;
inserting the introducer into the vascular system of the patient and steering a distal end of the introducer to the site;
inserting a shape forming element into a lumen of at least one of the electrode sheaths to form a bridging portion of a predetermined shape at the distal end of the catheter arrangement; and
urging the interconnected distal ends of the electrode sheaths out of a distal end of the introducer.

17: The method of claim 16 which includes, initially, inserting a stiffening element into a lumen of at least one of the electrode sheaths to facilitate steering of the catheter arrangement to the site.

18: The method of claim 17 which includes, once a distal end of the introducer is at the biological site, removing the stiffening element and replacing it with at least one shape forming stylet.

19: The method of claim 18 which includes, once the bridging portion of the catheter is exposed at the site, applying pressure to the bridging portion to urge electrodes carried by the bridging portion into contact with tissue at the site to enhance electrode-tissue contact.
